(19) United States
(12) Plant Patent Application Publication
(10) Pub. No.: US 2025/0107474 P1
(43) Pub. Date: Mar. 27, 2025

(54) BLACK RASPBERRY PLANT NAMED 'SHEKINAH TAHI'

(50) ;

(71) Applicant: Harvey HALL, Rotorua (NZ)

(72) Inventor: Harvey HALL, Rotorua (NZ)

(21) Appl. No.: 18/890,521

(22) Filed: Sep. 19, 2024

Related U.S. Application Data
(60) Provisional application No. 63/585,412, filed on Sep. 26, 2023.

(51) Int. Cl. A01H 6/74

(52) U.S. Cl. CPC A01H 6/7499
USPC PLT/204

(57) ABSTRACT

A new and distinct cultivar of spineless, floricane fruiting black raspberry ‘Shekinah Tahi’. Canes of ‘Shekinah Tahi’ have no spines, unlike the usual spiny canes of black raspberries. The spineless trait was introgressed from the original red raspberry source that originated in the old Scottish variety ‘Burnetholm’. The new cultivar fruits on laterals grown from buds of overwintered floricanes. ‘Shekinah Tahi’ produces large fruits in tight bunches on the ends of long spineless laterals.

### CROSS-REFERENCE TO RELATED APPLICATIONS

This application claims the benefit of U.S. Provisional Application No. 63/585,412, filed Sep. 26, 2023, the disclosure of which is incorporated by reference in its entirety.

### BACKGROUND OF THE INVENTION

The present invention relates to a new and distinct cultivar of spineless black raspberry. The new cultivar is distinguished from other black raspberry cultivars by being completely spineless in every part of the plant. Fruit is produced on floricanes giving mid-season production. ‘Shekinah Tahi’ fruit are too hard to pick for machine harvest, process production. ‘Shekinah Tahi’ fruit are too tightly bunched and not firm enough for fresh market commercial sales. ‘Shekinah Tahi’ offers significant opportunities for home gardeners as an easy to manage spineless variety. Traditional spiny black raspberries require considerable input for management and in the home garden ‘Shekinah Tahi’ will provide a welcome alternative.

### DESCRIPTION OF RELATED PRIOR ART

Black raspberries are well known for forming few or no suckers from their roots and new canes come from the basal crown of the plant. Traditional black raspberries are very spiny and they display a rambling growth because canes are arching and spreading, unlike the more upright growth of red raspberries. The spreading growth of black raspberries makes them difficult to manage, especially in a home garden situation. Traditional management practices to contain the plants involve cane tipping to reduce height, making the plants bushy, branching and low growing. When cane tips of the arching canes of black raspberries touch the ground, they form roots, especially in the Fall. A new plant is formed at each point where a cane tip produces roots, making them difficult to confine to one spot within a garden. A management practice of tipping black raspberries at about 1 m tall (around 3 feet) has been developed to limit tip-rooting and to promote significant branching and increased production of the new primocanes.

Hybrids between the black raspberry, Rubus occidentalis and the red raspberry, Rubus idaeus, are well known and are known as purple raspberries for their fruit color. Purple raspberries (Rubus neglectus L.) frequently do not tip root or produce suckers. In breeding of new black or red raspberry varieties, it is desirable to access traits from the other species. However, in practice it is difficult to breed traits from one species into the other and it takes multiple generations of breeding.

In red raspberries, the trait of fruit firmness was introgressed from the old black raspberry cultivar ‘Cumberland’. Dark color was the major obstacle to overcome to get back to acceptable red raspberry color.

When attempting to move traits from red raspberry into black raspberry there have also been challenges to get back to a true black color. When moving traits from red raspberry into black raspberries retention of purple fruit color is the obstacle. Incorporating the primocane fruiting trait into black raspberries took three generations, ending with backcrossing to black raspberry to produce ‘Ohio's Treasure’ (US PP27,871 P3). This process was alleviated by the dominant inheritance of the primocane fruiting trait.

The introduction of the gene for spinelessness from red raspberry into black raspberry was made significantly more difficult because its inheritance is recessive. For expression of this trait, it is necessary to incorporate two copies of the spineless gene into the diploid plant. To get a spineless black raspberry spineless selections were crossed with black raspberry. Selections from these hybrids were then inbred to expose the trait by back-crossing, selfing, or sib crossing to see the expression of the trait. ‘Shekinah Tahi’ results from around an eight-generation sequence of crosses, selfing and back crossing to black raspberry. The process began with crosses between ‘Munger’ black raspberry and the spineless red raspberry selections SCRI 689/81 and SCRI 6921/21 in 1969 and 1970. SCRI 689/81 and SCRI 6921/21 themselves were 4 and 5 generations of breeding from the old red raspberry cultivar ‘Burnetholm’, the source of the genetically spineless trait. Hence ‘Shekinah Tahi’ is 12-13 generations away from ‘Burnetholm’. ‘Shekinah Tahi’ was selected in 2010, over a period of 40 years since the original black raspberry cross in Scotland and as much as 65 years since the selection of AR1, a spineless selection derived from selfing ‘Burnetholm’. ‘Burnetholm’ was reported to carry genetic spinelessness by D. Lewis in 1939 and the trait was linked with eglandular cotyledons, where glandular hairs were absent on the cotyledonary leaves of spineless plants (FIG. 7). AR1, the spiny selections 3A/69 and 3B/45 and their spineless derivatives S25/8, S29/97 and S29/122 were produced in the 1950s. A range of spineless red raspberry cultivars has been developed in Scotland, England and around the world.

Named varieties of black raspberry have been cultivated since the 1800's, firstly with selections collected from the wild and then later from open pollinated seedlings and deliberate crosses. Almost 200 clones were mentioned in Hedrick's “The Small Fruits of New York” in 1925, with the majority of them being collected from the wild or from chance seedlings. Around twenty varieties resulted from deliberately grown seedling populations, and only six were the result of a deliberate crosses, the majority being from open pollinated seed lots. From all the selections mentioned in Hedrick's book in 1925, only three have been retained to the present day, ‘Cumberland’, ‘Munger’ and ‘Plum Farmer’, all from the 1890s, with ‘Munger’ being still the cultivar of choice for machine harvest, process production in Oregon. The genetic diversity of black raspberry Rubus occidentalis L. described in 1925 included black, yellow, orange and white fruited types, with or without bloom on the fruits and canes, and it also included two spineless varieties, Davison (1859) and Fay (1874). No record exists of these two varieties being used for breeding and it is assumed that the varieties have been lost.

Until the advent of more recent collections and their study the black raspberry was believed to have limited genetic diversity. However, limited genetic diversity was shown to be solely amongst the present range of cultivars and there was found considerable diversity amongst wild black raspberries. (Michael Dossett, Nahala Bassil, Kim Lewers and Chad Finn 2012: Genetic Diversity in wild and cultivated black raspberry (Rubus occidentalis L.) evaluated by simple sequence repeat markers—Genetic Resources and Crop Evolution 59. 10.1007/s10722-012-9808-8). In the absence of the diversity as found in wild collections, and as represented in The Small Fruits of New York in 1925, the spineless trait and the floricane fruiting traits have been introgressed from red raspberry into the black raspberry through the work initiated by Derek Jennings in Scotland and carried out by Harry Swartz in Maryland respectively.

In red raspberries the tendency in recent years has been to develop dual cropping varieties that bear a crop on the tips of the new primocanes and another crop on the floricanes in the following spring and summer. Economically this has become important as there are two production periods on the same variety and a spread of the marketing of the fruit. In black raspberry the production of cultivars bearing a primocane crop and a floricane crop has also been a desirable breeding objective and the primocane trait was sought from red raspberries, leading to the development and release of ‘Ohio's Treasure’ USPP27,871 P3, a variety with small (1-2.6 g), rather soft fruit. The primocane fruiting habit also has been developed from purely black raspberry genetics, with crosses between selections of wild types, leading to the release of the two black raspberry cultivars ‘Explorer’ and ‘Niwot’. ‘Explorer’ has since been discarded due to the lack of self-fertility and Niwot is being recognised for its primocane and floricane production.

‘Jewel’ from the New York State Agricultural Experiment Station in Geneva New York, part of Cornel University has large fruit for a variety solely derived from Rubus occidentalis L. black raspberry. The origin of ‘Jewel’ is reported to be Dundee x (Bristol x Dundee) in the 1973 release notice. However, in “Genetic Diversity in wild and cultivated black raspberry (Rubus occidentalis L.) evaluated by simple sequence repeat markers” this origin is questioned, and the derivation is suggested to be from ‘Huron’ or ‘Rachel’, and ‘Dundee’. Plants of ‘Jewel’ are vigorous, erect, winter hardy, consistently productive and canes are very spiny, typical of black raspberries. Fruiting is mid-season and fruit of ‘Jewel’ are firm, glossy and flavorful. ‘Jewel’ appears to be more disease resistant than most other black raspberries and it has slight susceptibility to powdery mildew and resistance to anthracnose. Fruit of ‘Jewel’ have a moderately tight cluster at the tip of fruiting laterals, and it exhibits concentrated ripening and a short production period. Sun scald of the plant and fruit may be a problem during hot weather conditions.

### BRIEF SUMMARY OF THE INVENTION

The present invention relates to a new and distinct cultivated variety (cultivar) of black raspberry, unique because of its genetically stable, completely spineless canes. The new cultivar is distinguished from other black raspberry cultivars by its completely spineless canes and black fruit. This is unlike the usual purple raspberries, from the hybrid origin, of a black-red raspberry cross. ‘Shekinah Tahi’ fruits on long, totally spineless, floricane laterals having tight clusters of fruit at lateral tips and some individual or paired fruits at the lateral leaf nodes, resulting in a moderately long season crop of large attractive black fruit. ‘Shekinah Tahi’ fruit have moderate firmness, making them unsuitable for shipping or for a long shelf life in the commercial market chain. However, they will last well for a home gardener or a U-Pick operation. Fruit release from the receptacle with moderate retention strength, making ‘Shekinah Tahi’ unsuitable for machine harvest-process production, with best quality fruit only being picked by hand harvest. ‘Shekinah Tahi’ produces a high yield of large fruit, and combined with the spineless canes makes the variety highly suited for the home garden producer. The spineless, vigorous, and semi-upright canes of ‘Shekinah Tahi’ also have a reduced ability to tip root, producing less spawn of new plants than either traditional spiny black raspberries, or from the suckers which are usual with red raspberries. ‘Shekinah Tahi’ pruning and training may be handled in a similar way to red raspberry, with no need to tip prune, making it possible for tall canes to be grown on a trellis and tipped when dormant during winter.

The following characteristics are outstanding:

1. All canes, laterals and fruiting pedicels are completely spine free, having two copies of the recessive ss gene for spinelessness, giving spinelessness in the diploid plants of ‘Shekinah Tahi’. In contrast ‘Jewel has very spiny canes, laterals, leaf pedicels, and fruit pedicels. This makes plants of ‘Shekinah Tahi’ much easier to manage than ‘Jewel’ or any other spiny black raspberry.

2. Vigorous growth of new primocanes with large, healthy green leaves, glaucous stems, and upright growth habit. Plants have stronger vigour than ‘Jewel’ and a more upright growth habit. ‘Jewel’ plants have smaller leaves, lower vigour, and a less upright growth habit. Mature primocanes have an outstanding appearance of the primocanes with excellent, attractive spineless and glaucous stems, with a purple-red background and a distinct silvery glaucus coating. This gives ‘Shekinah Tahi’ potential to be used for foliage for the cut flower and bouquet industry.

3. ‘Shekinah Tahi’ has a mid-season harvest, similar to ‘Jewel’.

4. ‘Shekinah Tahi’ displays excellent adaptation and good performance throughout the USDA hardiness zones 3-8 and in New Zealand from 37-41°S. ‘Shekinah Tahi’ is very hardy and has shown little or no winter damage in trials at Whately, Massachusetts.

5. Fruit size of ‘Shekinah Tahi’ is frequently larger than ‘Jewel’ in New Zealand and in Massachusetts, and consistently of a heavier weight, up to 4.5 g, due to the thick drupelet walls of ‘Shekinah Tahi’ fruit. Drupelet number in fruits of ‘Shekinah Tahi’ is also frequently higher than in fruit of ‘Jewel’.

6. ‘Shekinah Tahi’ fruit are borne in very tight clusters, predominantly at the lateral tips, with fruit frequently firmly held against their neighbors. ‘Jewel’ also is known for tight clusters of fruit, but the clusters are not as tight as with ‘Shekinah Tahi’ and it is less common for neighboring fruit to be touching or closely held against each other.

7. Fruit of ‘Shekinah Tahi’ has medium firmness, a little softer than with ‘Jewel’ and yields are also slightly lower, possibly due to the tight clustering.

8. Fruit of ‘Shekinah Tahi’ has a definite but mild black raspberry flavor, not quite as rich or fragrant as the fruit from ‘Jewel’.

9. Fruit of ‘Shekinah Tahi’ has a deep black color with a little more dustiness than with ‘Jewel’ which has fruit that are also a deep black color, but distinctly shinier.

10. With spiny black raspberries including ‘Jewel’ their spines and their arching growth habit necessitate significant management inputs to keep the plants in check and to manage their growth. The absence of spines with ‘Shekinah Tahi’ makes cane management, pruning and picking considerably easier than with traditional spiny types. ‘Shekinah Tahi’ pruning and training may be handled in a similar way to red raspberry, with no need to tip prune, making it possible for tall canes to be grown on a trellis and tipped when dormant during winter.

11. Propagation of ‘Jewel’ is similar to traditional black raspberries, being done by tip layering or with tissue culture. However, with ‘Shekinah Tahi’ tip layering is not as easy as with ‘Jewel’, with less cane growth making it to the ground and with less cane tips producing plantlets by tip rooting than with traditional black raspberries. Propagation by tissue culture is very effective and this is a worthwhile means for production of daughter plants.

### Origin of the New Cultivar

The new cultivar of black raspberry arose from the original importation to New Zealand of open pollinated seed from Dundee, Scotland of a selection named SCRI 8232C9 which was from the cross of the two spiny selections SCRI 7861D13 x SCRI 7860D8. SCRI 7861D13 was from the cross Munger x SCRI 7562B10, a first backcross to black raspberry. The spineless selection SCRI 7562B10 came from the cross of SCRI 7251/31 (Munger x SCRI 6921/21) x SCRI 7346/3 (Munger x SCRI 689/81). SCRI 7860D8 was from the cross Munger x SCRI 7560C11, another first backcross to black raspberry. The spineless selection SCRI 7560C11 came from the cross of SCRI 7251/56 (Munger x SCRI 6921/21)×7346/3 (Munger x SCRI 689/81). In New Zealand two selections were chosen from the open pollinated seedlings produced from SCRI 8232C9, V23 and V32. Each of these selections was crossed with ‘Jewel’ black raspberry to produce a further generation of breeding improvement, the populations 88407 and 88407R from intercrossing ‘Jewel’ and V23 and the populations 88408 and 88408R from intercrossing ‘Jewel and V32 yielding 4 and 7 selections respectively, one of which was released as the variety Ebony in New Zealand, with purple fruit. In the following generation 8 selections were intercrossed, giving a further 11 selections. In 1995 seedlings from crosses between red raspberries were grown on the Hall family farm at Woodstock, New Zealand, and amongst them was a spineless black raspberry with black fruit of unknown origin which was designated ‘Woodstock Black’.

‘Shekinah Tahi’ is a species hybrid with mostly black raspberry, Rubus occidentalis L. background, but with added traits from red raspberry Rubus idaeus L. including genetic spinelessness, the recessive gene s designating spine-free growth, improved uprightness and increased fruit size. The process by which these traits have been moved into the black raspberry is known as introgression which was begun by crossing the black raspberry seed parent ‘Munger’ with the spineless red raspberry pollen parents SCRI 6921/21 and SCRI 689/81. In the first generation of seedlings all plants were spiny due to the dominant effect of the spiny genes from the black raspberry ‘Munger’. In the second generation of breeding crosses between selections from the first generation gave seedlings which segregated for expression of spinelessness (ss) and spininess (genotypes Ss and SS) as designated by D Lewis in 1939 in his paper “Genetical studies in cultivated raspberries, I: inheritance and linkage” published in Journal of Genetics Vol. 38 Pages 367-379. Dominant spininess was represented by a capital S and the recessive spineless trait was represented by the lower case s, which needed to be present in a homozygous condition (ss) for the character to be expressed. Thus the process continued with backcrosses to the black raspberries ‘Munger’ (SS) and ‘Jewel’ (SS), giving spiny seedlings in the first generation, followed by an inbreeding generation to allow the spineless trait (ss) to be expressed.

University of Arkansas spineless blackberries have genetic spinelessness through an analogous gene derived from the diploid (2×) blackberry species, Rubus ulmifolius var inermis. In breeding from Rubus ulmifolius var inermis tetraploid (4×) blackberries were produced by crossing with a spiny tetraploid wild type, and this was inbred to produce a spineless variety known as ‘Merton Thornless’. ‘Merton Thornless’ was crossed with USA tetraploid blackberries to produce the varieties ‘Smoothstem’ and ‘Thornfree’, and ‘Thornfree’ formed the basis for breeding all the spineless varieties from the Arkansas breeding program and other programs around the world. With tetraploids the inheritance of the homozygous gene s (ssss) in breeding has different segregation ratios from diploid (2×) black and red raspberries, making it even more difficult to select transgressive segregants bearing the trait advancements desired for breeding improvement.

In each generation of breeding a predominating effect was occurring known as “linkage drag”, where, due to the small-tight chromosomes “crossing over” events were limited and genuine change was hard to achieve. However, these changes are needed to shift spinelessness into a plant bearing black raspberry fruit but expressing the spineless trait. In the case of transferring the primocane fruiting trait from red raspberry into the black raspberry ‘Ohio's Treasure’ the process was simplified by the dominant inheritance of that trait, and a primocane fruiting essentially black raspberry cultivar was achieved within three generations of breeding. However, in the development of ‘Shekinah Tahi’ what was sought was for the recessive gene conferring the spineless trait to be transferred from its position on a red raspberry chromosome into the analogous position in the black raspberry genome. This process was held back at each generation of crossing, inbreeding and backcrossing by a range of other traits being carried forward from the red raspberry background. The introgression process was further complicated due to the necessity of the crossing over event needing to happen in both chromosomes bearing the spiny and spineless gene loci, so that undesirable traits from red raspberry would be left behind.

In the case of ‘Shekinah Tahi’, the introgression of the spineless trait into black raspberry was held back (linkage drag) by the purple color of the fruit typical of black raspberry x red raspberry hybrids being carried from one generation to the next. The breakthrough in getting a spineless derivative with black fruit was achieved by the selection of the chance seedling that was designated ‘Woodstock Black’, having both spineless growth and the desirable black fruit color.

In the process of introgression of black fruit color back into black raspberries linkage drag worked in favour of the variety development process through carrying some desirable traits along with the purple fruit color which was eventually lost. These have included improved fruit size and an improved cane growth uprightness into the new hybrid black raspberries.

Bulk seedlings derived from ‘Woodstock Black’ and ‘Jewel’ were grown in 2009 and in 2010 the selection RZ10022 was chosen for its very large black fruit, spineless canes and semi-upright growth habit. This selection has been multiplied vegetatively in New Zealand and assessed for its production and qualities, and has been designated ‘Shekinah Tahi’ for this patent application.

### Asexual Propagation of the New Cultivar

‘Shekinah Tahi’ has been asexually propagated by tissue culture in Auckland, New Zealand and grown on in a commercial nursery in Nelson, New Zealand for assessment trials from 2010. No aberrant plants have been observed through all this asexual propagation and all plants have displayed similar spineless canes, growth and fruiting capabilities. Plants established in tissue culture without difficulty and multiplication in culture has been easy to achieve.

### BRIEF DESCRIPTION OF THE DRAWINGS

FIG. 1 shows a single fruiting lateral at the time of the ripening of the first fruit. Note the tightly clumped fruit at this stage of ripeness.

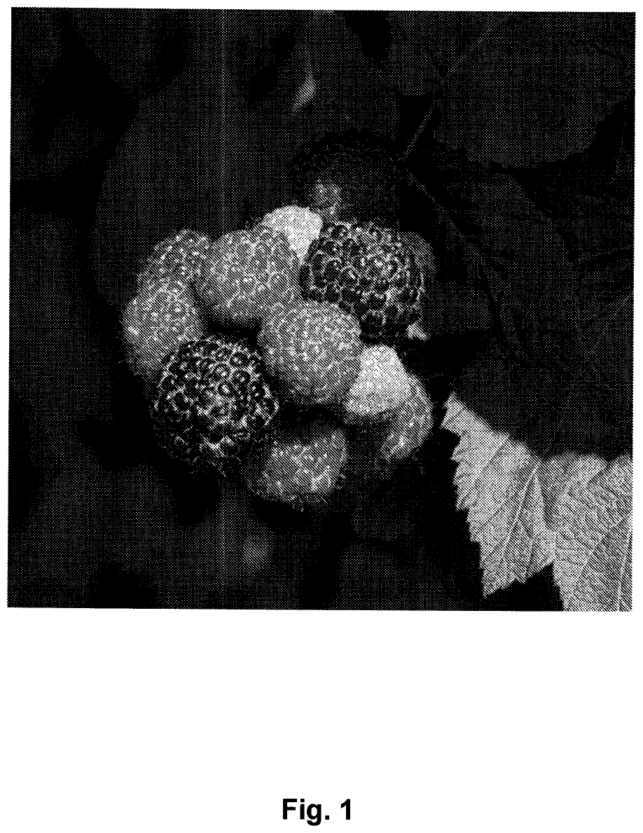

FIG. 2 shows a single fruiting lateral at a later stage of ripeness. Ripe fruit are still tightly clumped but are further separated than at the stage of first ripe fruit.

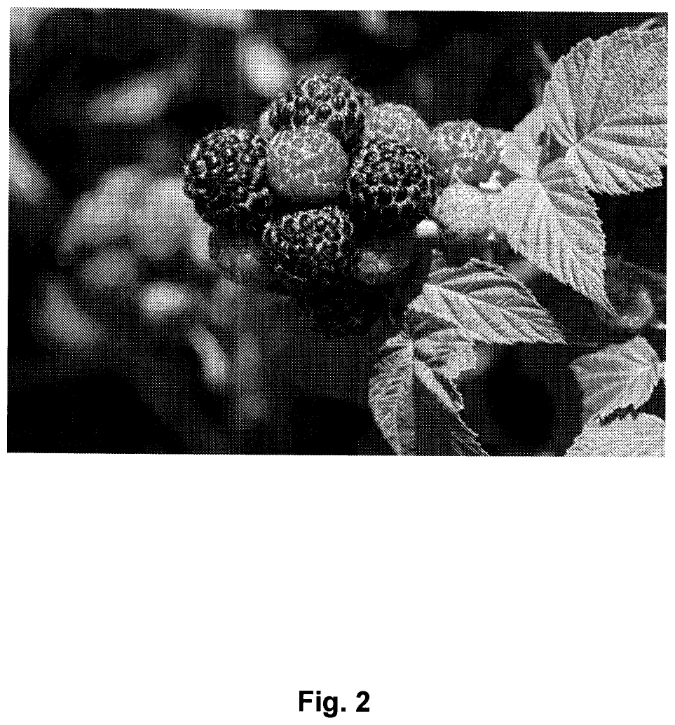

FIG. 3 shows several fruiting laterals with ripening fruit.

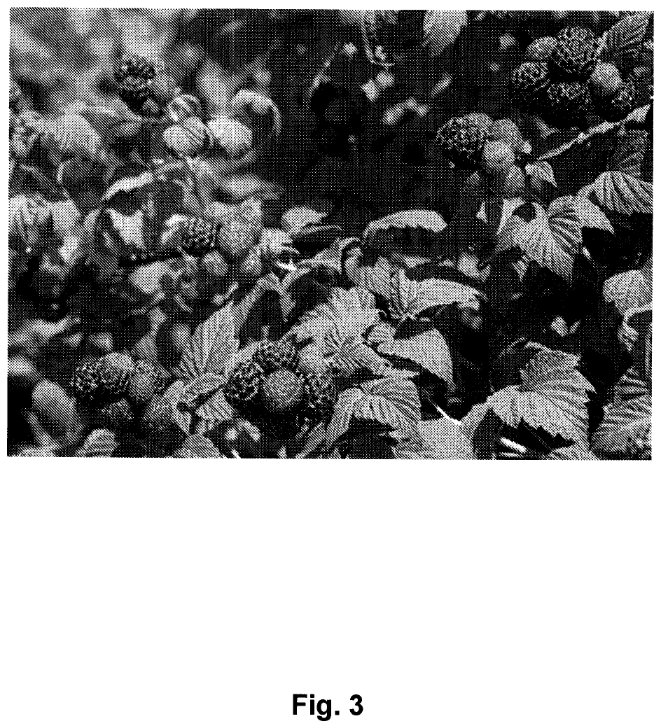

FIG. 4 shows a view of several canes and their fruit from further back.

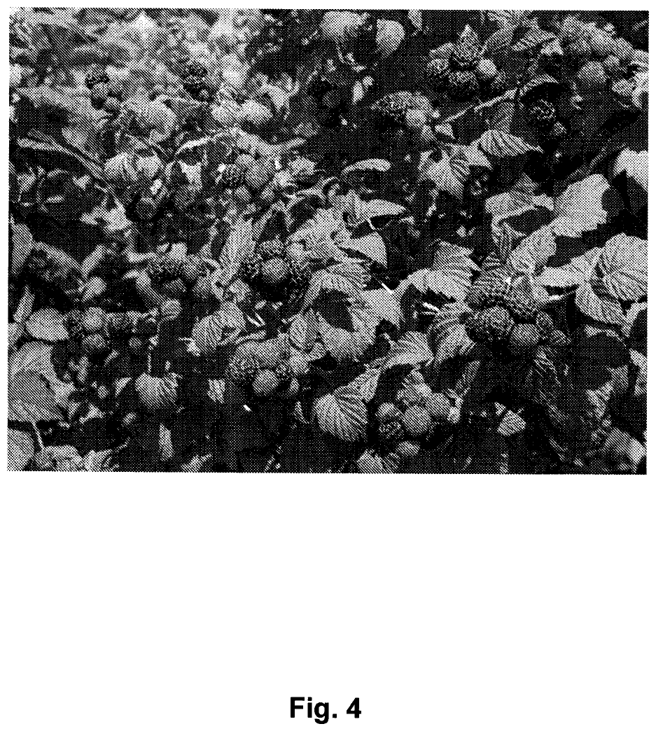

FIG. 5 shows a punnet of large and the high drupelet number and drupelet thickness of harvested fruit.

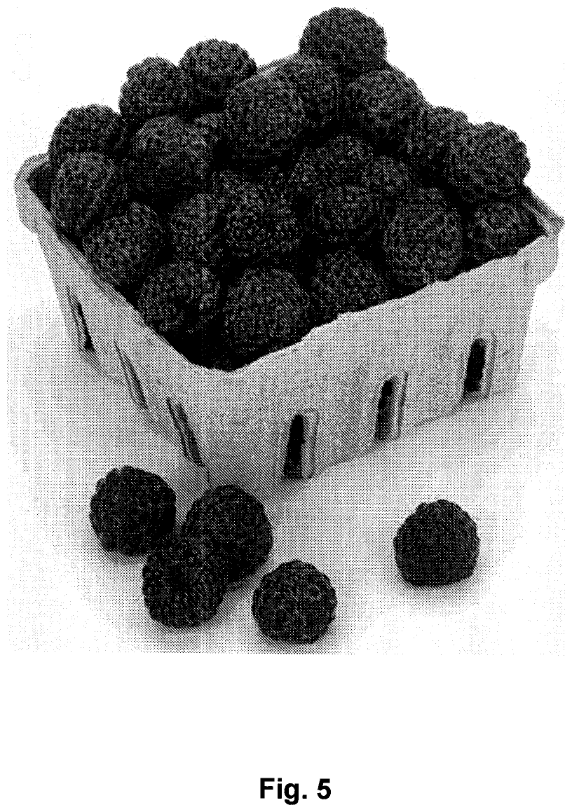

FIG. 6 shows a cane tip totally without spines and a view of the leafiness of new canes.

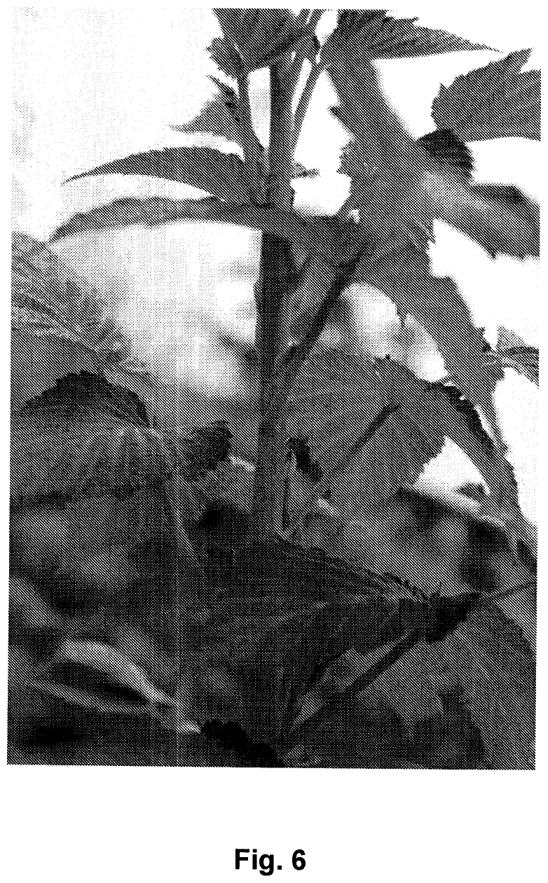

FIG. 7 shows cotyledonary leaves of spiny and spineless red raspberry as portrayed by D. Lewis in his 1939 paper: “Genetical studies in cultivated raspberries, I: inheritance and linkage” published in Journal of Genetics Vol. 38 Pages 367-379 as represented in Plate VIII, image 5.

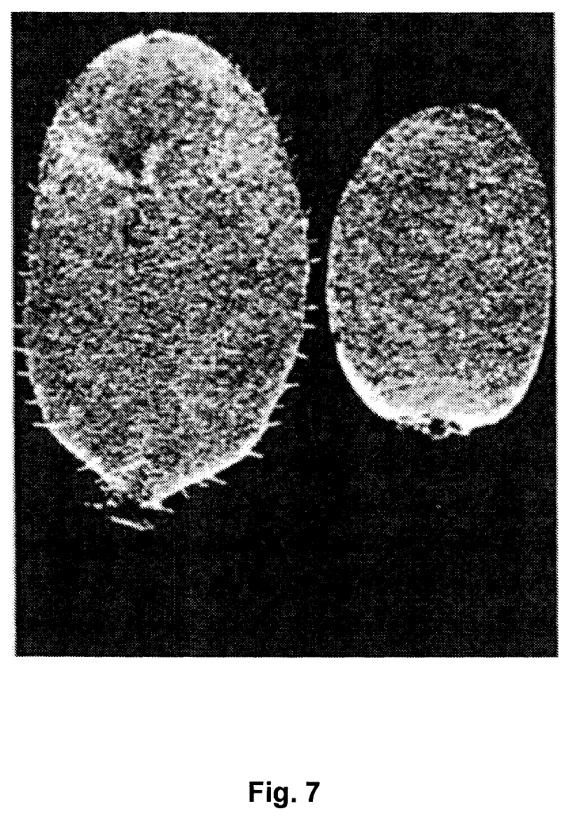

### DETAILED DESCRIPTION

The black fruited cultivar ‘Shekinah Tahi’ contains a genetic complement from red raspberry, as expressed in the genetic spinelessness, but its genetics are predominantly from black raspberry. On this basis the black fruited cultivar ‘Shekinah Tahi’ is botanically classified as the species Rubus occidentalis L.

The following is a detailed description of ‘Shekinah Tahi’ based on outdoor performance in Nelson province, New Zealand, and tunnel house performance in the Waikato and Bay of Plenty (other provinces in New Zealand). These observations have been extended and detailed by performance in Whately, Massachusetts where USA trials have been carried out. Observations and measurements have been made on plant morphology, fruit production and on post-harvest performance.

Primocane growth and appearance: New cane growth of ‘Shekinah Tahi’ is almost exclusively from crown suckers but an occasional root sucker may be found, although certainly not often enough so that root suckers may be used for propagation of plants to bulk up numbers quickly. First year primocane growth is completely spineless and growth is entirely vegetative until after plants enter dormancy during fall and at the onset of winter. Primocanes of ‘Shekinah Tahi’ may be tipped as is usual with black raspberries for home garden or commercial cultivation, but it also may be managed without cane tipping and pruned and trained like a red raspberry, which makes it easier to tie the canes to a trellis and to keep the plant confined in a home garden situation. ‘Shekinah Tahi’ may sometimes propagate itself through tip rooting but this behavior is inhibited by keeping the new canes elevated above the ground.

‘Shekinah Tahi’ propagates readily by tissue culture and, as described with ‘Niwot’, plant growth is rejuvenated (rendered juvenile again) by this process. New plants from tissue culture in their first year tend to be more spreading than when they mature in year two and onwards.

### Detailed Primocane Descriptions:

Spininess: Spines (prickles) are completely absent and self pollinated seedlings all have eglandular cotyledons and grow completely spineless, similar to the spineless red raspberries produced in Scotland and around the world and to the spineless blackberries from University of Arkansas and in other locations bearing spineless genes from Rubus ulmifolius var inermis.

Habit: Upright to semi-upright.

Emergence: Typically at the same time of ‘Jewel’ in the when planted together.

Length: Variable, according to the growing environment, age of the plant, the time of season planted and management. Primocanes of plants that have come through the previous winter in good condition will frequently exceed 2 m growth during the subsequent growing season.

Quantity: Usually 6-8 canes grow per crown, although this may be less or more under amended growing conditions.

Color: Glaucous. Primocanes have strong, glaucous, waxy coating that is easily removed by contact.

Cross section: Nearly circular.

Diameter: Estimated 1-1.5 cm at the base of canes.

Rachis: For apex leaflet, length typically 3-5 cm, totally devoid of spines or prickles.

Disease: No significant disease resistance or susceptibility observed compared with other black or red raspberries at Whately, Massachusetts.

Pests: No significant pest resistance or susceptibility observed compared with other black or red raspberries at Whately, Massachusetts.

Heat: ‘Shekinah Tahi’ has not displayed susceptibility to heat damage in conditions at Whately, Massachusetts.

Cold: ‘Shekinah Tahi’ has not displayed susceptibility to cold damage in conditions at Whately, Massachusetts. Plants appear to be hardy throughout USDA hardiness zones 3-8. The variety is very hardy with little to no winter damage being seen in the trials.

Primocane fruiting: absent

### Detailed Floricane Descriptions:

Floricane growth and appearance: Second year growth, known as floricane growth, begins with mid-season bud break and leads to flowering and fruiting in the same production season as ‘Jewel’.

Cane Color: Glaucous; cane with waxy bloom.

Laterals: Length variable from 20 to 75 cm from the top to lower laterals.

Leaves: Leaves grow alternately on the lateral, and are composed of 3 pinnately compound leaflets. The center leaflet is usually three-lobed.

Rachis: For apex leaflet, length typically 3-5 cm, totally devoid of spines or prickles.

Productivity: The plants of ‘Shekinah Tahi’ are productive, with almost the yield of ‘Jewel’.

Flower: Unscented, form the same as black raspberries.

Inflorescence: Long floricane laterals with flowers bunched at the tips, flowering at a similar time to ‘Jewel’.

### Fruit:

Size: Berries are very large for black raspberries with some berries being over 4.5 g in weight.

Drupelets: Mean count of drupelets per berry appears higher than other black raspberries. Acidity: ˜pH 3.6 Titratable acidity.

Sweetness: Soluble Solids brix measurement ˜12.

Color: black-purple when ripened.

The following characteristics distinguish ‘Shekinah Tahi’ from other black raspberries:

1) ‘Shekinah Tahi’ plants are entirely spineless and no spines have been observed on these plants at any time.

2) ‘Shekinah Tahi’ fruits are larger than all black raspberry varieties available to date, and fruit weights are up to 4.5 g

3) Fruit of ‘Shekinah Tahi’ are medium firm, regular and rounded and appear to have a higher drupelet number than other black raspberries

4) A tightly clustered fruit presentation on the end of fruiting laterals can make early picking difficult but the lack of spines makes picking and pruning this variety easier compared to traditional black raspberry varieties.

5) Canes appear to have stronger vigor than other black raspberries.

6) Growth habit appears to be more erect than other black raspberries.

1. A new and distinct spineless black raspberry plant known as ‘Shekinah Tahi’ described herein, illustrated and identified by the characteristics presented herein.
